(19) United States
(12) Patent Application Publication
(10) Pub. No.: US 2025/0035607 A1
(43) Pub. Date: Jan. 30, 2025

(54) Method for evaluating the hydrogen content in a steel sheet

(71) Applicant: ArcelorMittal, Luxembourg (LU)

(72) Inventor: Thomas DIEUDONNE, Semécouirt (FR)

(21) Appl. No.: 18/716,687

(22) PCT Filed: Dec. 6, 2022
(86) PCT No.: PCT/IB2022/061809

§ 371 Date: Jun. 5, 2024

(30) Foreign Application Priority Data
Dec. 17, 2021 (WO) PCT/IB2021/061904

(51) Int. Cl. G01N 33/2025; C21D 1/26; C21D 1/74

(52) U.S. Cl. CPC G01N 33/2025; C21D 1/26; C21D 1/74; C21D 2211/001; C21D 2211/002; C21D 2211/005; C21D 2211/008

(57) ABSTRACT

A method for evaluating the hydrogen content in a steel sheet while being submitted to an annealing process, including the following steps: estimating the microstructure of the steel sheet according to the temperature curve, computing the solubility of the hydrogen C_{H}, computing the volume concentration of trapped hydrogen in dislocations C_{T} and the volume concentration of hydrogen in interstitial sites of the crystal lattice C_{L}, calculating the hydrogen content Cₜₒₜₐₗ=C_{L}+C_{T} at each time step of the annealing process and outputting the hydrogen content Cₜₒₜₐₗ at each time step to a user.

The present invention relates to a method for evaluating the hydrogen content in a steel sheet while being submitted to an annealing process.

### BACKGROUND

A steel sheet is made of grains in which atoms are arranged in crystal lattice, thus forming the microstructure of the steel. Spaces between these atoms are called interstitial sites. The arrangement of atoms is not totally regular, and some arrangement defect can occur, which is the case for dislocations which are linear defect.

During the heat treatment of a steel sheet, hydrogen atoms present in the atmosphere of the furnace, can easily penetrate the steel and can be absorbed. Indeed, hydrogen can diffuse into the crystal lattice due to its atomic size of the same order of magnitude as the size of the interstitial sites of the crystal lattice. Hydrogen atoms may progressively diffuse and be trapped inside the defects such as dislocations.

The introduction and diffusion of hydrogen in the steel sheet is one of the mechanism responsible of the brittleness of the steel sheet, which could lead for example, to cracks formation along grain boundaries and/or dislocations gliding planes.

Because of hydrogen, steel strip can suffer ductility lost, also called hydrogen embrittlement.

### SUMMARY OF THE INVENTION

A purpose of the invention is to provide a method for evaluating the hydrogen content in a steel sheet undergoing an annealing process, and to output the hydrogen content at any time to a user.

A method for evaluating the hydrogen content in a steel sheet undergoing at least one annealing process following a thermal path, in which the temperature can be measured by sensors in a furnace having an atmosphere comprising hydrogen, said steel sheet comprising grains in which atoms are arranged in a crystal lattice, thus forming the microstructure of the steel including dislocations and interstitial sites, said method comprising the following successive steps:
determining the microstructure of the steel sheet as a function of the thermal path,
computing the solubility of the hydrogen C_{H}, at the surface of the steel sheet as a function of the microstructure, the temperature T and the hydrogen partial pressure p_{H2},
computing the volume concentration of trapped hydrogen in dislocations C_{T} and the volume concentration of hydrogen in interstitial sites C_{L}, as a function of the temperature T, of trapping rate of hydrogen k, of detrapping rate of hydrogen p, of C_{H} and of the microstructure,
calculating the hydrogen content Cₜₒₜₐₗ=C_{L}+C_{T} at any time of the annealing process, and
outputting through a computer display the hydrogen content Cₜₒₜₐₗ at any time to a user.

Hereinafter, C_{L} designates the concentration of hydrogen in the interstitial sites of the crystal lattice of the steel sheet and C_{T} the concentration of trapped hydrogen in the steel sheet. Dislocations are the only trapping sites considered in the invention, homogeneously distributed in the microstructure.

### BRIEF DESCRIPTION OF THE DRAWING

The invention will now be described in detail and illustrated by examples without introducing limitations, with reference to the appended figures:

FIG. 1 represents the temperature curve of processes P0 and P1,

FIG. 2 represents the temperature curve of process P2,

FIG. 3 illustrates the cells used in one embodiment of the method of the invention, to represent the microstructure at point F₁ of process P1

FIG. 4 represents the time evolution of C_{L}, C_{T} and Cₜₒₜₐₗ=C_{L}+C_{T} of P0,

FIG. 5 represents the time evolution of C_{L}, C_{T} and Cₜₒₜₐₗ of P1,

FIG. 6 represents the time evolution of C_{L}, C_{T} and Cₜₒₜₐₗ of P2,

FIG. 7 represents the temperature curve of process P3

FIG. 8 represents the temperature curve of process P4

FIG. 9 represents the time evolution of C_{L}, C_{T} and Cₜₒₜₐₗ of P3,

FIG. 10 represents the time evolution of C_{L}, C_{T} and Cₜₒₜₐₗ of P4,

FIG. 11 illustrates the cells used in one embodiment of the method of the invention, to represent the microstructure at point E₃ of process P3.

### DETAILED DESCRIPTION

The method according to the invention deals with the evaluation of the diffused hydrogen content of a steel sheet undergoing at least one annealing process.

During this annealing, the sheet is subjected to at least one heating step and one cooling step, according to a thermal path. Usually, heat treatments can be performed in an oxidizing atmosphere, i.e. an atmosphere comprising an oxidizing gas being for example: O₂, CH₄, CO₂ or CO. They also can be performed in a neutral atmosphere, i.e. an atmosphere comprising a neutral gas being for example: N₂, Ar or He. Finally, they also can be performed in a reducing atmosphere, i.e. an atmosphere comprising a reducing gas being for example: H2 or HNx.

In a preferred embodiment, the thermal path can also include at least one isothermal holding step, that can usually be preceded by a heating step and followed by a cooling step. The cooling step can comprise an isothermal holding, called an overaging sub-step followed by a subsequent cooling step. A hot-dip coating step in a hot metal bath can also be used during such thermal path and is another type of isothermal holding as the metallic sheet dipped in such hot metal bath will be maintained at the bath temperature during its retention time in such bath.

Said annealing can be, for example, recrystallization annealing, recovery or a tempering, and can be followed by these heat-treatments;
quench and tempering,
quench and partitioning.
a. The thermal path according to the invention corresponds to the successive temperatures T, heating and cooling rates and time spent in each section of the annealing process and optional subsequent heat-treatments.

The temperatures of the thermal path can be measured by sensors during the annealing and optional subsequent heat-treatments, or through calculations done with the use of a software.

The first step of the method according to the invention is to determine the microstructure of the steel sheet as a function of the thermal path of the annealing process, through for example calculations done with the use of a software like COMSOL®. The microstructure can also be determined thanks to sensors in furnace able to measure the austenite content in the steel, like X-CAP®.

In the frame of the present invention, the evolution of the microstructure occurs instantaneously only at certain points, corresponding to a phase change at given temperatures. The solubility of hydrogen C_{H} at the surface of the steel sheet is then calculated. The solubility of hydrogen is the aptitude of hydrogen to be dissolved in the steel sheet. This solubility depends on the temperature, partial pressure of hydrogen and on the phases present in the microstructure of the steel sheet. It can be calculated through the following equations [1] and [2] that will be described.

For a temperature T below or equal to Ac3, and preferably from 280K to 1184K:

$$log ( {C}_{H} ) = 0.5 log ( {p}_{{H}_{2}} ) - 3 - \frac{1500}{T} \qquad [ 1 ]$$

In the first part of the temperature curve where heating takes place, ferrite is the main structure in the steel sheet. The solubility of hydrogen C_{H} in ferrite is adequately calculated using above formula [1].

In the last part of the temperature curve where cooling takes place, part of the austenite formed above Ac3 can transform in bainite and/or martensite, depending on the composition of the steel and on the cooling rate. In that part of the curve, the solubility of hydrogen C_{H} in bainite and martensite is assumed to be the same as in ferrite and can be obtained adequately using also above formula [1].

For a temperature T above Ac3, preferably from 1184K to 1667K:

$$log ( {C}_{H} ) = 0.5 log ( {p}_{{H}_{2}} ) - 2.9 - \frac{1490}{T} \qquad [ 2 ]$$

In the middle part of the temperature curve where holding at high temperature can take place, it is assumed that austenite is the main phase in the steel sheet, and the solubility of hydrogen C_{H} in austenite is adequately calculated using formula [2] above.

In both equations [1] and [2], C_{H} is expressed in at % and p_{H2} is the hydrogen partial pressure in the furnace, expressed in Pa. These both equations are from the publication of Fromm & Jehn “Hydrogen in Elements”, (Bull. Alloys Phase Diagrams, 5 (3), 323-326 (1984)).

Determining the solubility of hydrogen C_{H} at the surface of the steel sheet is required as an input for the next step of the method according to the invention, wherein the volume concentration of hydrogen in the interstitial sites of the crystal lattice C_{L} and the volume concentration of trapped hydrogen C_{T} in the steel sheet, both expressed in mole of hydrogen by m³ of iron (mol_{H}/m³_{Fe}), are computed with numerical simulations through the resolution of the following equations:

$$\frac{\partial {C}_{L}}{\partial t} + \frac{\partial {C}_{T}}{\partial t} \qquad [ 3 ]$$

$${\partial }^{2} C ⊤ \frac{\partial {C}_{T}}{\partial t} = \frac{k}{{N}_{L}} {C}_{L} ( {N}_{T} - {N}_{A} \times {C}_{T} ) \qquad [ 4 ]$$

$$\text{?}$$

$$\text{?} \text{indicates text missing or illegible when filed}$$

The different parameters and constants of both equations [3] and [4] will now be explained.

D_{L} is the diffusion coefficient in the crystal lattice, expressed in m²/s, which depends on the temperature and phases present in the steel sheet at that temperature. This diffusion coefficient expresses the aptitude of hydrogen to diffuse inside a material. The higher the coefficient, the more easily the hydrogen diffuses.

In ferrite, martensite and bainite this coefficient diffusion D_{L} of hydrogen is assumed to be the same and is calculated through the following equation:

$${D}_{L} = 5.12 * {10}^{- 4} {e}^{- \frac{4150}{R T}} \qquad [ 5 ]$$

In austenite, the coefficient diffusion D_{L} of hydrogen is calculated according to the following equation:

$${D}_{L} = 5.8 * {10}^{- 3} {e}^{- \frac{53800}{R T}} \qquad [ 6 ]$$

R=8.314 J/mol·K being the universal gas constant and T the temperature expressed in K.

These equations show that at equivalent temperature, the hydrogen diffuses more easily in the ferrite, martensite and bainite than in austenite.

N_{L}=5.2×10²⁹ sites/m³ is the volume density of the interstitial sites in the steel sheet. In the method according to the invention, N_{L} is assumed to be the same in all the phases of the microstructure.

N_{T} is the volume density of dislocations, expressed in sites/m³. Dislocations have an associated trapping energy of E_{B}=27000 J/mol. In the method according to the invention, one dislocation can trap one or more hydrogen atoms. The volume density of dislocations N_{T} is calculated by using the surface density of dislocation ρ_{dis} expressed in sites/m², thanks to the following formula:

$${N}_{T} = α \frac{{ρ}_{dis}}{{a}_{bcc \frac{\sqrt{3}}{2}}} \qquad [ 7 ]$$

with α being the number of dislocations per Burger's vector, which represents the ability of dislocations to trap hydrogen atoms. The higher this coefficient, the more the dislocations trap the hydrogen atoms. a_{bcc} is the lattice parameter in the bcc structure expressed in angstroms. In the frame of the invention, this lattice parameter is the same in ferrite, martensite and bainite which are all bcc structures. In a preferred embodiment, those parameters can take the following values:
α=7
a_{bcc}=2.87 Å

In the frame of the invention, atoms of hydrogen cannot be trapped by dislocations in austenite, because of their low diffusion coefficient.

k and p are respectively the hydrogen trapping and detrapping rates, expressed in s⁻¹, corresponding to the quantity of hydrogen atoms respectively trapped and detrapped, as a function of time, defined by the following equations

$$k = {k}_{0} exp ( \frac{- {E}_{T}}{R T} ) \qquad [ 8 ]$$

$$p = {p}_{0} exp ( \frac{- {E}_{D}}{R T} ) \qquad [ 9 ]$$

E_{T}=4 150 J/mol, being the energy of trapping, which is the energy that hydrogen atom must provide to be trapped, and E_{D}=E_{T}+E_{B}=31150 J/mol being the energy that hydrogen must provide to be detrapped.
Constants k0 and p0 are the hydrogen trapping and detrapping coefficients expressed in s⁻¹. They are used as fitting parameters for the calculation of C_{T} and C_{L} together with N_{T} in the different phases of the microstructure. Such fitting parameters can be determined through a comparison between experiments performed on a given steel composition and calculations according to the invention, iterated until experimental and calculated values converge.

Finally, N_{A}=6.02×1023 mol⁻¹ is the Avogadro number.

As described above, some of the calculations performed in the frame of method of the invention depend on the phases present in the steel at a given point of the temperature curve. Moreover, equation [3] depend on the depth x of the steel portion for which the calculations are done. To give an accurate evaluation of the hydrogen trapped in the entire thickness of the steel sheet, it is preferred to consider that the sheet is made of the repetition of N cells of 5 μm×5 μm, in order to simulate at least part of the thickness of the sheet. In a preferred embodiment, half- of the thickness of the sheet is used, N being calculated through the formula:

$$N = thickness of the steel sheet / ( 2 ⋆ 5 μ m )$$

The other half thickness of the steel sheet behaves exactly like the first one and that the diffusion of hydrogen is homogeneous in the full length of the sheet.

C_{H} values can then be calculated using equations [1] and [2] all along the temperature curve. Such C_{H} values are then used as the C_{L} values for the first row of cells.

Formula [3] and [4] can be successively applied to each cell to finally provide the values of C_{T} and C_{L} for the full thickness of the sheet.

In the last step of the method according to the invention, the total hydrogen content Cₜₒₜₐₗ is determined by calculating the sum of C_{L} and C_{T} at any time, before to be output to a user through a computer display.

The invention will be now illustrated by the following examples, which are by no way limitative.

### Example 1

Cold rolled steel sheets having a composition consisting of 0.07% wt of C, 2.62% wt of Mn, 0.25% wt of Si, 0.3% wt of Cr, 0.16% wt of Al, 0.091% wt of Mo, the remainder of the composition being iron and unavoidable impurities resulting from the smelting, and a thickness of 1 mm are supplied.

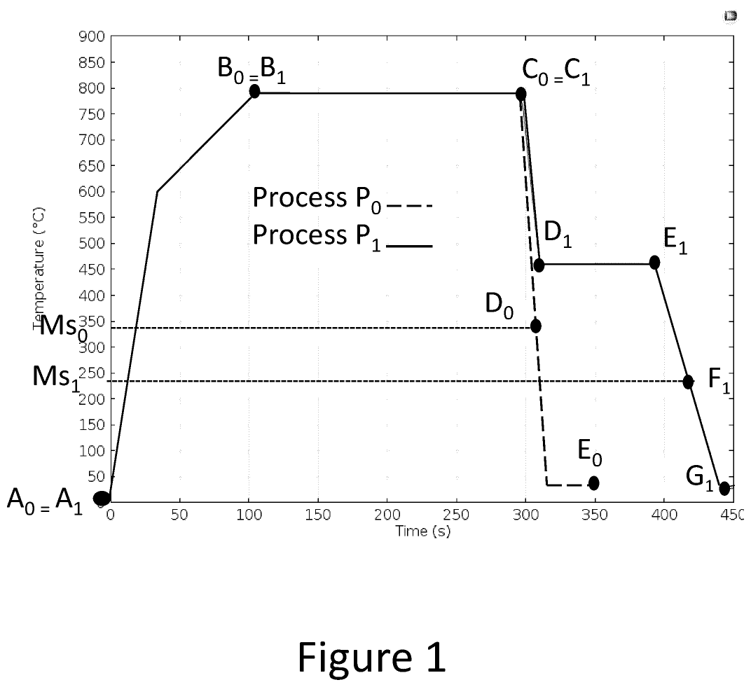

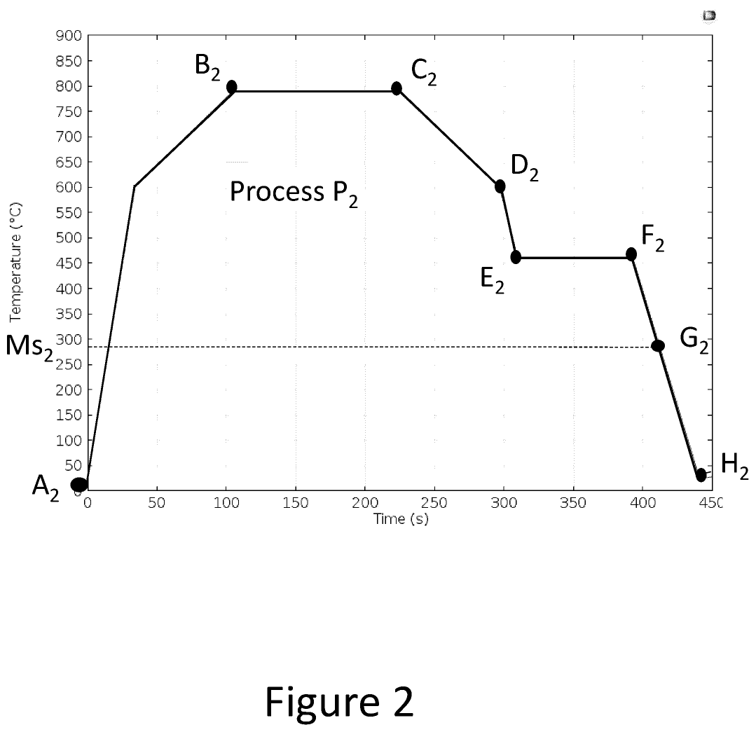

Such sheets can then undergo one annealing process among process P0, P1 and P2 as described in FIGS. 1 and 2. In all processes, the sheets are heated to a temperature T_{H} of 790° C., and maintained at said temperature for a holding time t_{H}, in a furnace having an atmosphere consisting of 5% of hydrogen, the rest being N₂, with a dew point of −50° C. In the process P0, the steel sheet is then cooled to room temperature (RT).

In the process P1, the steel sheet is cooled from T_{H} to a temperature T₁ of 465° C. and maintained at said temperature for a holding time t₁ of 86 s, before being cooled to room temperature at a cooling rate of 10° C./s.

In the process P2, the steel sheet is first slowly cooled from T_{H} to a temperature T₁ of 600° C. at a cooling rate of 2.6° C./s and secondly cooled from T₁ to a temperature T₂ of 465° C. at a cooling rate of 40° C./s. The steel sheet is maintained at said temperature T₂ for a holding time t₂ of 86 s, before being cooled to room temperature (RT) at a cooling rate of 10° C./s.

The temperatures Ms in each process are obtained by dilatometry measurement.

Experimental trials have been performed with these process parameters in order to obtain the experimental values of hydrogen content. The following specific conditions were applied:

TABLE 1
Process parameters

| | | | First cooling step | | | Second cooling step | | | Third cooling step | | |
|---|---|---|---|---|---|---|---|---|---|---|---|
| | Heating | | | Cooling | | | Cooling | | | Cooling | |
| | T_{H} | t_{H} | T₁ | rate | t₁ | T₂ | rate | t₂ | Temperature | rate | |
| Process | (° C.) | (s) | (° C.) | (° C./s) | (s) | (° C.) | (° C./s) | (s) | (° C.) | (° C./s) | Ms (° C.) |
| P0 | 790 | 192 | RT | 40 | — | — | — | — | — | — | Ms₀ = 340 |
| P1 | 790 | 192 | 465 | 40 | 86 | RT | 10 | — | — | — | Ms₁ = 240 |
| P2 | 790 | 119 | 600 | 2.6 | — | 465 | 40 | 86 | RT | 10 | Ms₂ = 280 |

The experimental hydrogen content is measured thanks to TDA experiments at the end of the process. The steel sheets have been heated at a heating rate of 1200° C./h, in order to obtain an hydrogen thermal desorption.

The corresponding diffusible hydrogen contents are gathered in Table 2:

### Hydrogen Content Evaluated According to the Invention

TABLE 2
Experimental hydrogen content

| | Diffusible hydrogen |
|---|---|
| Process | (wt ppm) |
| P0 | 0.34 |
| P1 | 0.10 |
| P2 | 0.10 |

First, the microstructure of the steel sheet is estimated at each point (A0, B0, C0, D0, E0; A1, B1, C1, D1, E1, F1, G1; A2, B2, C2, D2, E2, F2, G2, H2) of the temperature curves as represented in FIG. 1 and FIG. 2.

The phase transformations of the microstructure occur instantaneously at the indicated points only. The estimated microstructures are gathered in Table 3:

TABLE 3
Estimated microstructures

| | | | | | Bainite |
|---|---|---|---|---|---|
| Process | Points | Ferrite(%) | Austenite(%) | Martensite(%) | (%) |
| P0 | A₀ | 100 | — | — | — |
| | B₀ | 30 | 70 | — | — |
| | C₀ | 30 | 70 | — | — |
| | D₀ | 30 | — | 70 | — |
| | E₀ | 30 | — | 70 | — |
| P1 | A₁ | 100 | — | — | — |
| | B₁ | 30 | 70 | — | — |
| | C₁ | 30 | 45 | — | 25 |
| | D₁ | 30 | 25 | — | 45 |
| | E₁ | 30 | 25 | — | 45 |
| | F₁ | 30 | — | 25 | 45 |
| | G₁ | 30 | — | 25 | 45 |
| P2 | A₂ | 100 | — | — | — |
| | B₂ | 30 | 70 | — | — |
| | C₂ | 30 | 70 | — | — |
| | D₂ | 30 | 45 | — | 25 |
| | E₂ | 30 | 25 | — | 45 |
| | F₂ | 30 | 25 | — | 45 |
| | G₂ | 30 | — | 25 | 45 |
| | H₂ | 30 | — | 25 | 45 |

During the heating step up to T_{H}, ferrite is the main phase until the temperature reaches AC1, where ferrite starts being transformed into austenite. During the holding step at T_{H}, respectively starting at points B₀, B₁ and B₂, the microstructure is then made of ferrite and austenite.

For process P0, during the cooling at room temperature, all the austenite is transformed into martensite at Ms0 temperature (point Do).

During the first cooling of process P1 beginning at point C1, a part of austenite is transformed into bainite. Austenite continues to be transformed into bainite during the subsequent holding step, starting at D1. The microstructure during the cooling starting at E1 is the same as in the step between D1 and E1. The austenite is finally transformed in martensite at point F1, corresponding to the Ms1 temperature.

In process P2, during the slow cooling starting at C2, no phase transformation occurs. A part of austenite is transformed into bainite during the subsequent cooling starting at D2. Austenite continue to be transformed into bainite during subsequent overaging starting at E2. The microstructure at F2 is the same as E2 and the austenite is transformed into martensite at point G2 at Ms2 temperature.

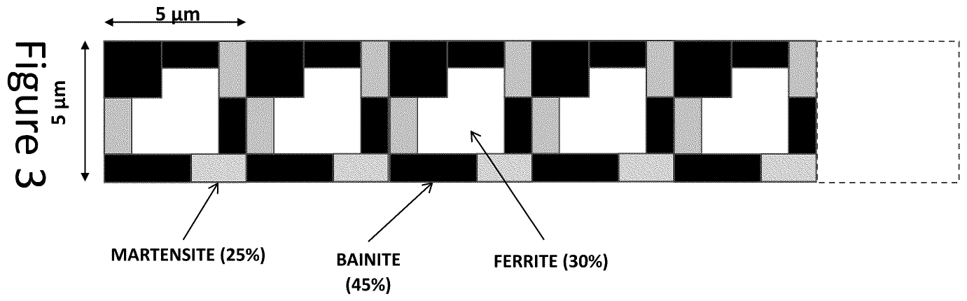

Half of the thickness of the steel sheet is simulated through the repetition of N=100 cells of 5 μm×5 μm. The phase percentages of table 3 are taken into account through the percentages of the surface of the cells, as illustrated on FIG. 3 corresponding to microstructure at point F₁: 25% of the surface of the cells represents the 25% of martensite inside the steel, 45% of the surface of the cells represents the 45% of bainite and 30% of the surface of the cells represents the 30% of ferrite inside the steel sheet.

C_{H} values are then calculated using equations [1] and [2] with a hydrogen partial pressure p_{H2} of 5066.5 Pa for all points where a phase transformation occurs. Such C_{H} values are then used as the C_{L} values for the first row of cells.

N_{T} and the trapping and detrapping coefficients were fitted, using the following protocol. Steel sheets having a composition according to example 1, have been heated at a temperature of 850° C. in a furnace having an atmosphere consisting of 5% of H₂, the rest being N₂, and maintained at said temperature for a holding time of 260 s, before being quenched. The experimental hydrogen content in each sheet has then been measured through TDA experiments, by heating the steel sheet at a heating rate of 1200° C./h. After several iterations, the best fitting parameters have been chosen as follows:
k₀=10⁵ s⁻¹
p₀=10² s⁻¹
N_{T} in ferrite=10²⁴ sites/m³
N_{T} in bainite=5 10²⁴ site/m³
N_{T} in martensite=10²⁵ sites/m³

The volume concentration of hydrogen in the interstitial sites of the crystal lattice C_{L} and the volume concentration of trapped hydrogen C_{T} in the steel sheet are then computed through the resolution of equations [3] and [4], taking into account the microstructures described above, and for each of the point where a phase transformation occurs all along the thermal curves.

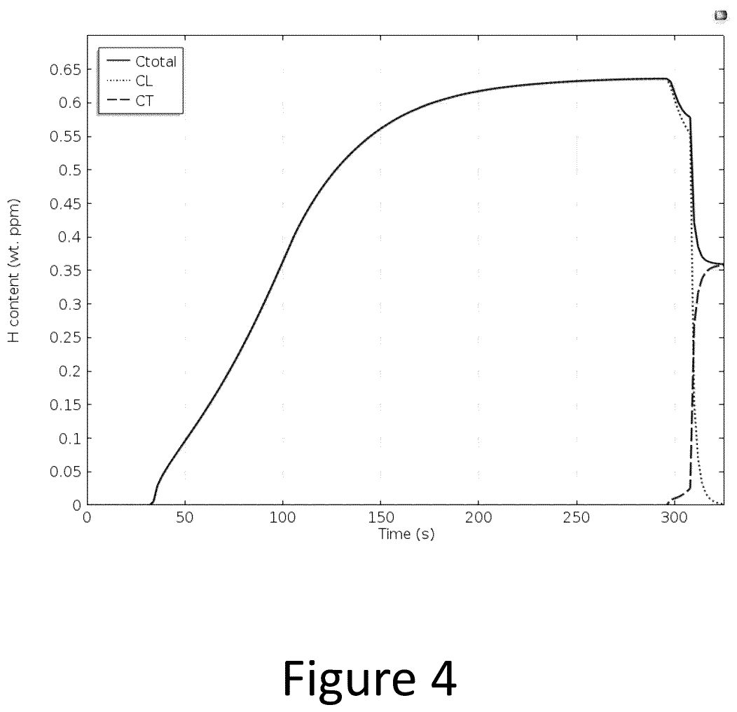

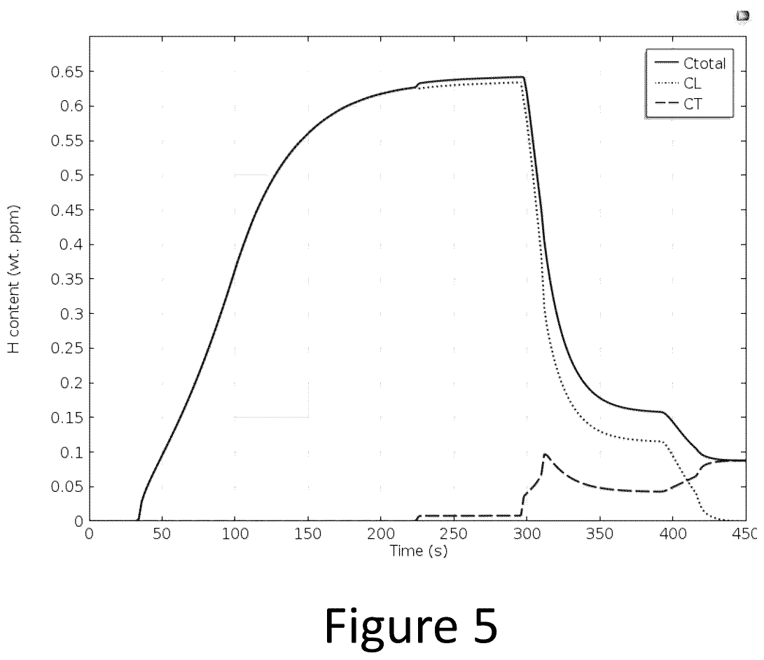

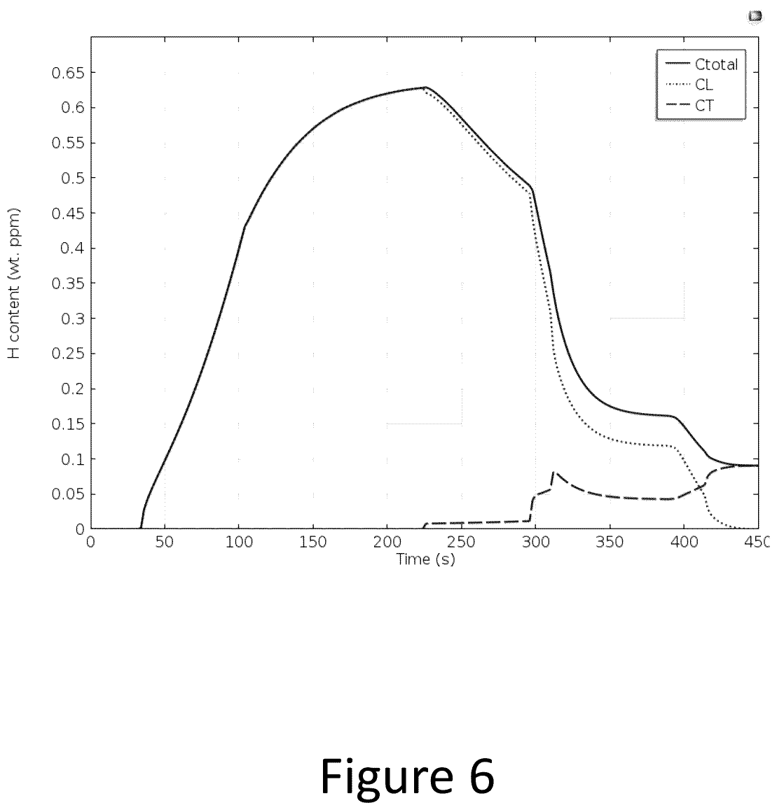

Cₜₒₜₐₗ (Cₜₒₜₐₗ=C_{L}+C_{L}) is then calculated at each of these points and transmitted to an operator. The respective curves gathering all points are shown on FIGS. 4 to 6 corresponding to processes P0, P1 and P2.

For process P0, the final hydrogen content as determined by the method according to the invention is 0.36 ppm, compared to 0.34 ppm measured experimentally at the end of the annealing process

For processes P1 and P2, the final hydrogen content as determined by the method according to the invention is 0.09 ppm, compared to 0.10 ppm experimentally measured.

As shown by these examples, the method according to the invention well predicts the hydrogen content evolution.

This shows that the method according to the invention can accurately evaluate at any time the hydrogen content, in order to be output to a user during the production of the steel sheet.

### Example 2

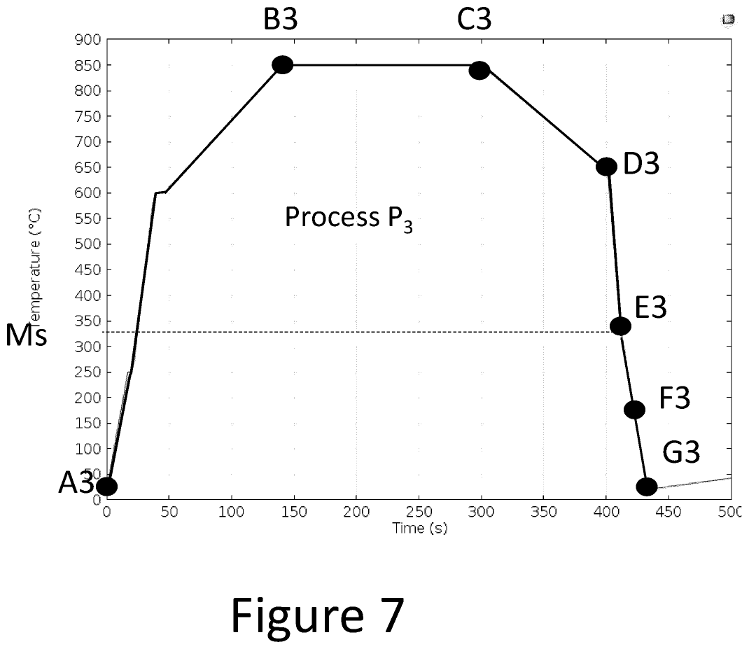

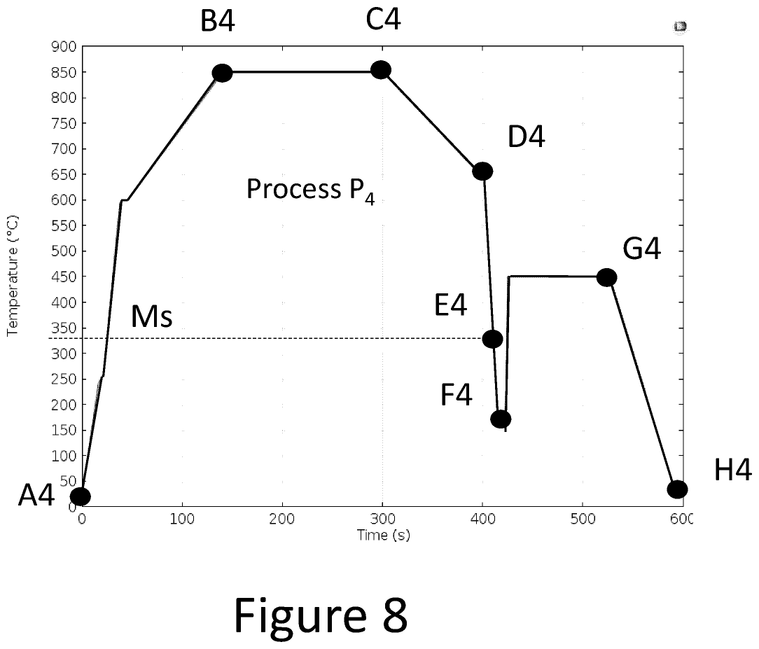

Cold rolled steel sheets having a composition of 0.19% wt of C, 3.86% wt of Mn, 1.27% wt of Si, 0.39% wt of Al, 0.2% wt of MO, 0.0235% wt of Nb, 0.0293% wt of Ti, the remainder of the composition being iron and unavoidable impurities resulting from the smelting, and a thickness of 1.2 mm are supplied. Such sheets can then undergo an annealing process among process P3 and P4 as described in FIGS. 7 and 8.

In all processes, the sheets are heated to a temperature T_{H} of 850° C., and maintained at said temperature for a holding time t_{H} of 159 s. The steel sheets are then cooled from T_{H} to a temperature T₁ of 600° C. at a cooling rate of 2° C./s before being quenched to a temperature T_{Q} of 170° C.

In process P3, the steel sheet is then cooled from T_{Q} to room temperature (RT).

In process P4, the steel sheet is then reheated from T_{Q} to a temperature Tₒ of 450° C., maintained at said temperature Tₒ for a holding time tₒ of 102 s before being cooled to room temperature.

The temperatures Ms of this grade in each process are obtained by dilatometry measurement.

Experimental trials have been performed with these process parameters in order to obtain the experimental values of hydrogen content. The following specific conditions were applied:

TABLE 4
Process parameters

| | | | First cooling step | | | | | | |
|---|---|---|---|---|---|---|---|---|---|
| | Heating | | | Cooling | | Overaging | | Cooling step | |
| | T_{H} | t_{H} | T₁ | rate | Quenching | Tₒ | tₒ | Temperature | |
| Process | (° C.) | (s) | (° C.) | (° C./s) | T_{Q} (° C.) | (° C.) | (s) | (° C.) | Ms(° C.) |
| P3 | 850 | 159 | 600 | 2 | 170 | — | — | RT | 330 |
| P4 | 850 | 159 | 600 | 2 | 170 | 450 | 102 | RT | 330 |

Trials with these process parameters have been performed in order to obtain the experimental values of hydrogen content.

### Table 5: Experimental Hydrogen Content

The experimental hydrogen content is measured thanks to TDA experiments at the end of the process, by heating the steel sheets at a heating rate of 1200° C./h.

The corresponding hydrogen contents are gathered in Table 5:

| Process | Diffusible hydrogen (wt ppm) |
|---|---|
| P3 | 0.38 |
| P4 | 0.07 |

### Hydrogen Content Evaluated According to the Invention

First, the microstructure of the steel sheet is estimated at each point (A3, B3, C3, D3, E3, F3, G3; A4, B4, C4, D4, E4, F4, G4, H4) of the temperature curves as represented in FIG. 7, and FIG. 8.

The phase transformation of the microstructure occur instantaneously at the indicated points. The estimated microstructures are gathered in Table 6:

TABLE 6
Estimated microstructures

| | | Ferrite(%) | Austenite(%) | Martensite(%) |
|---|---|---|---|---|
| P3 | A₃ | 100 | — | — |
| | B₃ | — | 100 | — |
| | C₃ | — | 100 | — |
| | D₃ | — | 100 | — |
| | E₃ | — | 0 | 100 |
| | F₃ | — | 0 | 100 |
| | G₃ | — | 0 | 100 |
| P4 | A₄ | 100 | — | — |
| | B₄ | — | 100 | — |
| | C₄ | — | 100 | — |
| | D₄ | — | 100 | — |
| | E₄ | — | 15 | 85 |
| | F₄ | — | 15 | 85 |
| | G₄ | — | 15 | 85 |
| | H₄ | — | 15 | 85 |

During the heating step up to T_{H}, ferrite is the main phase until the temperature reaches AC3, where ferrite is transformed into austenite. During the holding step at T_{H}, respectively starting at the point B₃ and B₄, the microstructure is made of 100% of austenite.

In process P3, at points C₃ and D₃, the microstructure is the same as the one of B₃. All the austenite is transformed into martensite at Ms temperature corresponding to E₃, and the microstructure is unchanged until the end of the process.

In process P4, at points C₄ and D₄, the microstructure is the same as the one of B₄. A part of austenite is transformed into martensite at Ms temperature corresponding to E₄, and the microstructure is unchanged until the end of the process.

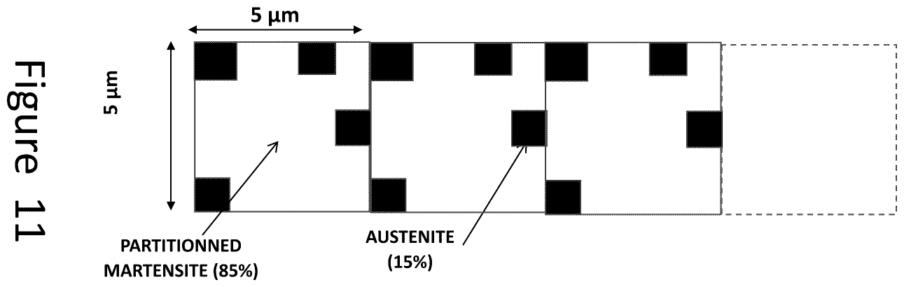

Half of the thickness of the steel sheet is simulated through the repetition of N=120 cells of 5 μm×5 μm. The phase percentages of table 5 are taken into account through the percentages of the surface of the numerical cells, as represented on FIG. 11 corresponding to microstructure at point E₄: 15% of the surface of the cells represents the 15% of austenite inside the steel, 85% of the surface of the cells represents the 85% of partitioned martensite.

C_{H} values are then calculated using equations [1] and [2] with a hydrogen partial pressure p_{H2} of 5066.5 Pa for all points where a phase transformation occurs. Such C_{H} values are then used as the C_{L} values for the first row of cells.

N_{T} and the trapping and detrapping coefficients were fitted, using the following protocol. Steel sheets having a composition according to example 2, have been heated at a temperature of 850° C. in a furnace having an atmosphere consisting of 5% of H₂, the rest being N₂, and maintained at said temperature for a holding time of 260 s, before being quenched. The experimental hydrogen content in each sheet has then been measured through TDA experiments, by heating the steel sheet at a heating rate of 1200° C./h. After several iterations, the best fitting parameters have been chosen as follows:
k₀=10⁵ s⁻¹
p₀=10² s⁻¹
N_{T} in ferrite=10²⁴ sites/m³
N_{T} in bainite=5 10²⁴ site/m³
N_{T} in martensite=10²⁵ sites/m³

The volume concentration of hydrogen in the interstitial sites of the crystal lattice C_{L} and the volume concentration of trapped hydrogen C_{T} in the steel sheet are then computed through the resolution of equations [3] and [4], taking into account the microstructures described above, and for each of the point where a phase transformation occurs all along the thermal curves.

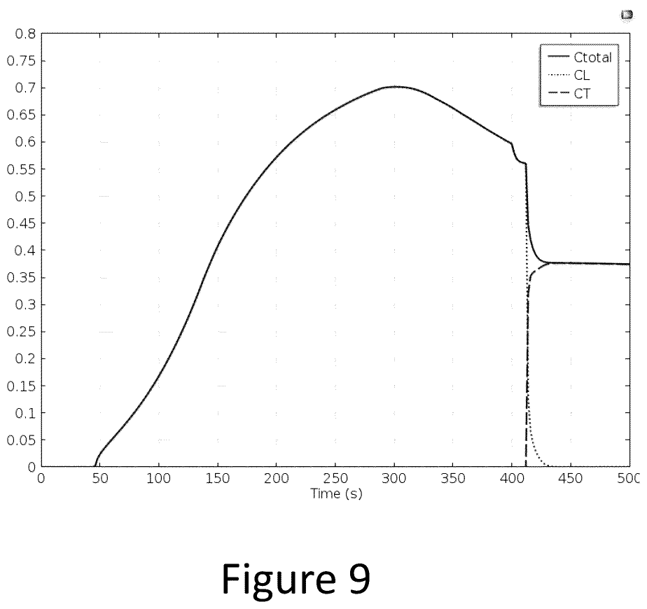

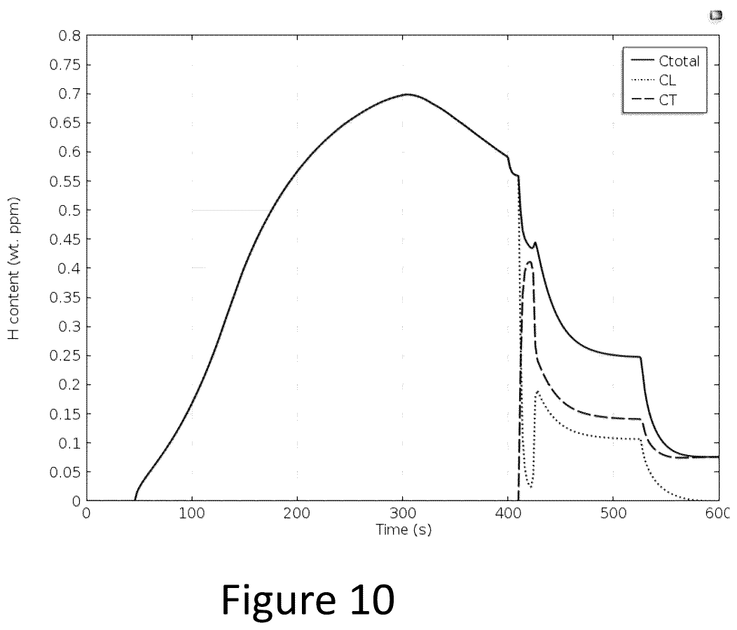

Cₜₒₜₐₗ (Cₜₒₜₐₗ=C_{L}+C_{L}) is then calculated at each of these points and transmitted to an operator. The respective curves gathering all points are shown on FIGS. 9 and 10 corresponding to processes P3 and P4.

As shown by these examples, the method according to the invention well predicts the hydrogen content evolution.

The numerical hydrogen content calculated at the end of the process P3 at point G₃, (t=430 s), is 0.37 ppm, compared to 0.38 ppm measured experimentally. At point H₄ (t=590 s) of the process P4, the numerical hydrogen content calculated is 0.07 ppm, as measured experimentally.

The hydrogen content can be output to a user at any time of the annealing process and the full curve can be output as well at the end of the annealing process.

What is claimed is:

1-6. (canceled)

7. A method for evaluating hydrogen content in a steel sheet undergoing at least one annealing process following a thermal path, a temperature T measureable by sensors in a furnace having an atmosphere comprising hydrogen, the steel sheet comprising grains in which atoms are arranged in a crystal lattice, thus forming a microstructure of the steel including dislocations C_{T} and interstitial sites C_{L}, the method comprising the following successive steps:
determining the microstructure of the steel sheet as a function of the thermal path;
computing a solubility of the hydrogen C_{H}, at the surface of the steel sheet as a function of the microstructure, the temperature T and a hydrogen partial pressure p_{H2};
computing the volume concentration of trapped hydrogen in dislocations C_{T} and the volume concentration of hydrogen in interstitial sites C_{L}, as a function of the temperature T, of trapping rate of hydrogen k, of detrapping rate of hydrogen p, of C_{H} and of the microstructure;
calculating the hydrogen content Cₜₒₜₐₗ=C_{L}+C_{T} at any time of the annealing process; and
outputting through a computer display the hydrogen content Cₜₒₜₐₗ at any time to a user.

8. The method as recited in claim 7 wherein C_{T} and C_{L} are calculated through the resolution of the following equations over at least part of the thickness of said steel sheet:
$$\frac{\partial {C}_{L}}{\partial t} + \frac{\partial {C}_{T}}{\partial t} \qquad [ 3 ]$$
$$\frac{\partial {C}_{T}}{\partial t} = \frac{k}{{N}_{L}} {C}_{L} ( {N}_{T} - {N}_{A} \times {C}_{T} ) \qquad [ 4 ]$$
D_{L} being the lattice diffusion coefficient of hydrogen in the crystal lattice,
x being the depth inside said steel sheet,
N_{L} being the volume density of interstitial sites,
N_{T} being the volume density of dislocations,
N_{A} being the Avogadro constant.

9. The method as recited in claim 7 wherein the solubility of the hydrogen C_{H} is calculated through the following equations:
for a temperature T below or equal to Ac3,
$$log ( {C}_{H} ) = 0.5 log ( {p}_{{H}_{2}} ) - 3 - \frac{1500}{T} \qquad [ 1 ]$$
for a temperature T above Ac3,
$$log ( {C}_{H} ) = 0.5 log ( {p}_{{H}_{2}} ) - 2.9 - \frac{1490}{T} \qquad [ 2 ]$$
p_{H2} being the hydrogen partial pressure in the furnace and C_{H} being expressed in at %.

10. The method as recited in claim 7 wherein the microstructure of the steel sheet includes at least one phase among ferrite, austenite, martensite and bainite and wherein the lattice diffusion coefficient of hydrogen D_{L} is calculated through the following equations
$${D}_{L} = 5.12 * {10}^{- 4} {e}^{- \frac{4150}{R T}}$$
in ferrite, martensite and bainite
$${D}_{L} = 5.8 * {10}^{- 3} {e}^{- \frac{53800}{R T}}$$
in austenite

11. The method as recited in claim 7 wherein the trapping rate of hydrogen k and detrapping rate of hydrogen p are calculated through the following equations
$$k = {k}_{0} exp ( \frac{- {E}_{T}}{R T} ) \\ p = {p}_{0} exp ( \frac{- {E}_{D}}{R T} )$$
$$\text{?}$$
$$\text{?} \text{indicates text missing or illegible when filed}$$
with k₀ and p₀ being trapping and detrapping coefficients, E_{T} being the trapping energy,
E_{D} being the detrapping energy, and R the universal constant gas.

12. The method as recited in claim 7 wherein the volume density of dislocations N_{T} is calculated through the following equation
$${N}_{T} = α \frac{{ρ}_{dis}}{{a}_{bcc \frac{\sqrt{3}}{2}}}$$
ρ_{dis} being the surface density of dislocation, α being the number of dislocations per Burger's vector, and a_{bcc} being the lattice parameter.

13. A method for manufacturing a steel sheet comprising using the method as recited in claim 7 and the hydrogen content Cₜₒₜₐₗ to alter the annealing process.
